(19) United States
(12) Patent Application Publication
(10) Pub. No.: US 2016/0165928 A1
(43) Pub. Date: Jun. 16, 2016

(54) METHODS OF REDUCING METHANE EMISSION DERIVED FROM RUMINAL FERMENTATION WHILE SIMULTANEOUSLY REDUCING RISK OF NITRATE INTOXICATION

(71) Applicant: Grasp Indústria e Comércio Ltda., Curitiba (BR)

(72) Inventors: Alysson Hoffmann Pegoraro, Curitiba Parana (BR); Rafael Canonenco de Araujo, Sao Paulo (BR)

(21) Appl. No.: 15/043,249

(22) Filed: Feb. 12, 2016

Related U.S. Application Data
(63) Continuation of application No. 14/119,245, filed on Nov. 21, 2013.

(30) Foreign Application Priority Data
May 23, 2011 (BR) PI1102284-0

(51) Int. Cl. A23K 1/175; A61K 9/50; A61K 33/06; A61K 45/06

(52) U.S. Cl. CPC A23K 1/22; A23K 1/1813; A23K 1/1753; A23K 1/1751; A23K 1/175; A61K 33/06; A61K 45/06; A61K 9/5015

(57) ABSTRACT

Methods of reducing methane emission in one or more ruminant animals, while simultaneously avoiding nitrate intoxication, are disclosed. The methods may include feeding the ruminant animal(s) a feed including a slow-release ruminant feed additive, where the slow release of nitrate ion from the feed additive keeps the overall levels of released nitrate in the rumen below a level that would result in nitrate intoxication, yet provides a level of non-protein nitrate ion to ruminant bacteria high enough to act as an antimethanogenic agent, resulting in a reduced production of methane. The slow-release ruminant feed additive may be in a granular or round form and encapsulated by a coating. The feed additive may include 40% to 85% in weight nitrate ion, 0% to 50% in weight sulfur, 1% to 40% in weight of a coating including hydrogenated vegetable fat; and 0% to 20% of additives in weight.

### CROSS REFERENCE TO RELATED APPLICATIONS

This application is a continuation of U.S. patent application Ser. No. 14/119,245, filed on Nov. 21, 2013, which was a U.S. National Stage patent application filed pursuant to 35 U.S.C. §371 of International Patent Application PCT/BR2012/000157, filed on May 23, 2012, which claims priority to Brazil Patent Application No. PI1102284-1, filed on May 23, 2011, where the content of each of these applications is hereby expressly incorporated by reference in their entireties for all purposes.

### FIELD OF THE INVENTION

The present invention is related to the field of livestock production, specifically to the field of animal nutrition, more specifically to the use of nutritional supplements and additives for ruminants, exactly to the use of nitrates and sulfates encapsulated with hydrogenated fats, used to reduce ruminal methane emission, in order to allow a slow-release of the active compounds in the rumen, maximizing their complete metabolism and reducing the risks of animal intoxication.

### BACKGROUND OF THE INVENTION

Greenhouse gases (GHG), mainly carbon dioxide (CO₂), methane (CH₄), and nitrous oxide (N₂O), partially absorb the infra-red radiation emitted by Earth's surface, which hampers its dissipation to the space. This process, however, is essential for the maintenance of life in Earth because hinders excessive heat loss and keeps the planet warmed. Notwithstanding, an increase in the GHG concentration magnifies this natural phenomenon, thereby resulting in the raise of global mean temperature, a process called global warming.

Taking into account that industrialization process and world's population show a tendency to increase in the next years, the agricultural sector has been pressured to become more efficient in terms of GHG emissions. Due to its shorter half-life (10 years) when compared with carbon dioxide (150 years) and nitrous oxide (150 years), methane mitigation plays a key role in the achievement of positive short-term climate effects derived from GHG mitigation.

In Brazil, methane generated by enteric fermentation represents 12% of total CO₂-eq (carbon dioxide equivalent) emitted by human activities, approximately. From this amount, 90% is represented by rumen fermentation. Considering only the agricultural sector, enteric fermentation corresponds to 53% of Brazilian agricultural CO₂-eq emissions. In global terms, methane produced by ruminants represents around 22% of total methane produced by human activities.

Methane is naturally produced during microbial fermentation in the rumen, being rumen the first stomach of a ruminant—an anaerobic fermentation chamber where cohabits different kinds of microbes inside, such as bacteria, protozoa, fungi, bacteriophages etc. Methane generation is essential for the maintenance of microbial processes, although methane production is always referred as an energy loss for the animal, ranging from 5 to 12% of gross energy intake.

Methane is produced by methanogenic Archaea, a population that consumes CO₂ and H₂ as substrates for energy production and eliminates methane as an end-product. In the rumen, methane production is necessary to keep a low hydrogen pressure, which is necessary for the processes of microbial fermentation responsible for feed degradation, basically cellulose, hemicellulose, starch, sugars, protein, peptides, aminoacids etc.

Ruminal interspecies hydrogen transfer is defined as the process when Archaea consume hydrogen disposed by the metabolic activities of other rumen microorganisms. When hydrogen is not eliminated from the rumen as methane, it occurs an increase in the hydrogen pressure that results in overall inhibition of microbial fermentation.

For instance, dairy cows produce about 500 L/day of CH₄, which corresponds to 357 g/day, approximately. Brazilian researches determined that dairy cows kept on pasture produce around 278 to 403 g/day of methane.

Basically, there is two ways of methane mitigation:

a) to stimulate metabolic pathways that are able to compete with methanogenesis, being examples the utilization of acetogenic microorganisms, organic acids (malate, fumarate etc), and hydrogen acceptors (hydrogen peroxide, nitrates, sulfates etc);

b) to reduce ruminal hydrogen production, being examples the use of ionophores (e.g. monensin sodium), essential oils, and plant secondary compounds.

Besides the mentioned techniques, other potential strategies to reduce ruminal methane production are defaunation (elimination or reduction of protozoa), inoculation of live yeasts, control of Archaea population by immunization or vaccination, and nutritional strategies such as supplemental fats and an increase of concentrate feeds (e.g. grains) in the diet.

So far, all techniques to mitigate methane present limitations. Some of them show only transitory effects that disappear over time (e.g. essential oils, tannins, monensin, vaccines etc), while others show variable results (e.g. essential oils, tannins, saponins, vaccines etc). Moreover, some substances may be toxic to animals (e.g. some chemicals used to eliminate protozoa, chloroform, and high doses of unprotected and readily available nitrates), may not be viable due to elevated costs (e.g. organic acids), or having their use prohibited (e.g. ionophores such as monenin sodium, salinomycin, and lasalocid sodium in Europe). Finally, some techniques are too incipient, being examples the vaccination, immunization, and inclusion of acetogenic microorganisms.

Nitrate salts (NO₃⁻) have a higher affinity to H₂ when compared with CO₂, allowing nitrate-reducing microorganisms to compete with methanogenic Archaea for substrate. The reduction of nitrate to nitrite (Equation 1) and its further reduction to ammonia (Equation 2) generate more energy than the reduction of CO₂ to methane (Equation 3). This greater energy production provides a competitive advantage towards nitrate-utilizing microbes in comparison with methanogenic Archaea.

NO₃⁻+2H⁺→H₂O+NO₂⁻ (Eq. 1; ΔG₀=−130 kJ/mol of hydrogen)

NO₂⁻+4H₂→.NH₄⁺+2H₂O (Eq. 2; ΔG₀=−124 kJ/mol of hydrogen)

CO₂+4H₂→CH₄+2H₂O (Eq. 3; ΔG₀=−16.9 kJ/mol of hydrogen)

According to Equations 1 and 2, each mol of nitrate reduced to ammonium avoids the production of 1 mol of methane. In addition, similarly to urea, ammonium originated from nitrate metabolism serves as a N source for microbial protein synthesis. Consequently, there is a potential of using nitrate as a non-protein nitrogen (NPN) and, at the same time, anti-methanogenic agent. As a result, urea normally used as a NPN source in diet formulation for ruminants can be replaced by nitrate, combining the nutritional and anti-methanogenic potential to the diet.

Researches have showed that methane produced by rumen fermentation was reduced by 46.6% when using unprotected (uncoated) source of nitrate.

Nitrates when fed without prior adaptation—sudden inclusion—are toxic to animals including ruminants, causing a disease denominated methemoglobinemia. This disease is well-recognized in the field, being observed, as example, when animals ingest drinking water with high nitrate concentrations or when fed forages, mainly from temperate climates, that accumulated high levels of nitrate.

Once ingested, nitrate is metabolized by ruminal microorganisms to its intermediate compound, the nitrite (Equation 1). By a second reaction, nitrite is reduced sequentially to ammonium (Equation 2). The first reducing-reaction which leads to nitrite formation occurs in a rate faster than the reaction that consumes nitrite. As a consequence, there is a ruminal nitrite accumulation, with nitrite being the toxic compound for the animal. Nitrite is readily absorbed by the wall of digestive tract and passes to blood circulation, converting the ferrous form of hemoglobin (Fe²⁺) to the ferric form (Fe³⁺). The ferric form is unable to transport oxygen to the tissues, resulting in death caused by anoxia—privation of O₂. In general, symptoms are a rapid pulse rate and an increased respiration rate, followed by muscular tremors and general weakness. Membranes of eyes, mouth, and nose become a darker color due to oxygen deficit, with blood showing a brownish or “chocolate” pigmentation. Death occurs in extreme situations. In a chronic situation, the disease results in loss of performance (lower milk production, body weight gain, wool production).

It is well established that gradual adaptation of ruminants to nitrate allows multiplication and increase in the activity of nitrate-reducing microorganisms, mainly Selenomonas ruminantium subsp. lactilytica, Veillonella parvula, Wolinella succinogenes, and Megasphaera elsdenii, thereby reducing the risks of nitrite accumulation. However, the adaptation of animals to nitrate brings some practical and operational problems to the ruminant production system. Dietary changes stress the animals, lowering the productive potential of animals during this period. Moreover, adaptation periods are potentially dangerous due to mistakes and errors caused by handlers during ration preparation and offering of feed to the animals. Similarly to nitrate, the reduction of sulfate (SO₄²⁻) to sulphydric acid (H₂S) are also an alternative route to sink hydrogen and to minimize the ruminal production of methane (Equation 4). In the rumen, similarly to the methanogenic Archaea, sulfate-reducing bacteria utilize hydrogen for their growth. As a result, stimulating the growth of sulfate-utilizing microorganisms is a strategy to reduce methane, thus enhancing an alternative pathway of hydrogen consumption. The energy production derived from sulfate reduction (ΔG₀=−152 kJ) is higher than the energy resulted from methane production (ΔG₀=−131 kJ), allowing this alternative metabolic pathway to compete with methanogenesis.

SO₄²⁻+4H₂+2H⁺→H₂S+4₂O (Equation 4)

The use of a sulfur source is especially important to minimize the risks of intoxication by nitrate. Sulfur is reduced to H₂S, which acts as a hydrogen donator for the reduction of nitrite to ammonium. As a consequence, less accumulation of nitrites means a lower risk of intoxication. It is widely known by the scientific community that sulfur compounds are able to reduce the risks of nitrate intoxication.

It is realized, therefore, a gap in the art related to animal nutrition, of products that reduce methane emission without being harmful to animals, e.g. risks of intoxication, or being convenient to apply and use, not demanding high investments or, in addition, complex processes.

Based on this, and thinking on an uninterrupted development of products, it is proposed an innovation, at present claiming the privileges of its protection by its novelty and inventive activity, as exposed as follow. It is proposed, therefore, an encapsulated nutritional additive, in a granular form, thereby allowing the slow-release of nitrate and sulfates, and variations on its composition.

Such granules, or their variations, are manufactured with nitrates and sulfates, which are responsible by the mitigation of methane, and additives, or also similar compositions, coated/encapsulated with vegetable fats that are responsible for the reduction of releasing rate and solubilization of this salts in the rumen environment, with the purpose of avoiding animal intoxication and promoting the complete metabolism of nitrate and sulfates in the rumen.

In a similar way, alternatively to coating with vegetable fats, it is possible to use any other material compatible with the animal nutrition that shows equal or similar properties from those presented in fats in terms of promoting a controlled release of the substance. It is distinguished here natural materials, degradable in the rumen or not, such as cellulose and carboxycellulose-based emulsions (added, as example, with calcium carbonate, saccharose, vegetable oils, and xanthan gum), coatings containing starch and other polysaccharides mixed with polyvinyl alcohols, as well as coatings based on lignin/lignosulphonates or chitosan biopolymers. Alternatively, coating may also be composed of synthetic polymers, degradable in the rumen or not, such as carboxyvinyl; polyacrylic acid (acrylic resins, polyethylenes etc); alginates; polyhydroxyalkanoates; polyhydroxyoctanoates; polyhydroxybutyrates (Biopols); polycaprolactones; polylactic acids; solutions of biuret with urethane and tungue oil; mixtures of isocyanates with alkydic resins, castor oil and peroxides; mixtures of stearamides with paraffin, magnesium stearate; other resins (polyurethanes, polyolefins, polyesthers, polyepoxides, silicones, polyvinylidene chloride etc, as well as mixtures thereof); alkyl and cycloalkyl amines; paraffins and waxes derived from petroleum.

Among the fats used for encapsulation, it is mentioned here soybean oil, castor oil, palm oil, cashew nut shell oil or cashew nut shell liquid, cottonseed oil, linseed oil, peanut oil, babassu oil, sunflower oil, coconut oil, canola oil, wheat oil, rice oil, corn oil, cocoa oil, safflower oil, and waxes (from vegetable or animal sources), being examples carnauba wax, corn wax, castor wax, and bee wax. Here, it is not excluded the isolated use of just one fat source, as well as the use of a combination of two or more than two fat sources, aiming at bringing advantages such as the supply of functional fatty acids, in terms of melting point, plasticity, waxy properties, as well as shock and abrasion resistance.

### ANALYSIS OF RELATED ART

The document WO010921 contemplates the reduction of gastro-intestinal methanogenesis in ruminants, with the utilization of agents able to compete with methanogenesis by hydrogen atoms during the normal fermentation of ingested feeds. The products are offered comprehending high amounts of a combination of one compound based on nitrate and one compound based on sulfate and, alternatively, probiotic microorganisms for the reduction of nitrite, as well as methods to reduce gastro-intestinal methanogenesis in ruminants by using such compositions. Such method does not consider the protection, coating, and encapsulation of nitrates and sulfates for a slow ruminal release, moving away from the proposed object characteristics.

The invention U.S. Pat. No. 6,231,895 describes the offering of nutritional supplements for ruminants with a level of non-protein nitrogen (NPN) which results in a controlled and safe release of ammonia under conditions of ruminal incubation. In another form, this invention provides a nutritional supplement for ruminants with controlled release of non-protein nitrogen which comprehends urea particles encapsulated with a coating made with a rumen-degradable polymer. This invention moves away from the object proposed here because does not deal with supplements based on nitrates and sulfates.

The document WO03068256 deals with methods and compositions for an improvement of ruminal fermentation efficiency, enhancing the efficiency of dietary starch utilization, avoiding a deleterious increase in ruminal concentration of lactic acid/or a drop on ruminal pH, as well as promoting the benefit growth of ruminal microorganisms. Methods and compositions of the present invention can also include supplementation with yeasts, buffer agents, ionophores, or other agents to stimulate growth and productivity; however it does not cite any coating based on fats, thus moving away from the characteristics of the object proposed here.

The patent PI0608919 demonstrates a structural element suitable to use in the manufacturing of a releasing device for the administration of a intra-ruminal active agent composed of a compact material in a ruminant animal, which comprehends a mixture of iron, graphite and, optionally, powdered copper, with graphite being present in the mixture in an amount from 2% to 7% in weight, the copper in an amount from 0% to 5% in weight, and iron in an amount between 88% to 98% in weight, in relation to the total weight of iron, copper and graphite. A variety of structural elements can be combined in order to achieve a structural unity of a releasing device. The patent describes a device for a slow ruminal release of a composition, and does not cite in its composition the use of nitrate or either the process of encapsulation, thus moving away from the characteristics of the innovation proposed here.

The patent PI0305047 consider a ration for ruminant animals composed mainly of starchy material from babassu nuts, which receives in its composition a mixture of urea, sulfur, babassu starch, babassu meal, in a proportion of 30% to 60%, 1.5% to 3.0%, 20% to 30%, and 20% to 30%, respectively. The process of compound preparation is comprehended by the stage of babassu nut selection, shelling of nuts, cleaning of starchy material, starch material grinding, product formulation, and thermal treatment. In this compound, NPN is protected by babassu starch, coated in a gelatinous form, which hampers solubilization in water. It also provides a slow ammonia release in the rumen, increasing, therefore, the utilization of NPN by rumen microorganisms during microbial protein synthesis. The compound is indeed a product that respects the N:S ratio of 10:1 and, besides providing protein to the ruminant, also provides energy which comes from starch. Using this product, intoxication risks are low and, in small quantities, it is possible to feed calves in creep-feeding system. The document is related to a composition based on starch and non-nitrate substances, moving away from the characteristics of the invention proposed here.

The document PI9201217 presents a slow-release capsule, adapted to be introduced in the rumen of an animal by its esophagus, kept inside the rumen for a long period for continuous liberation of the biological active composition held in the capsule. The capsule in a long and tubular-shape body, a tube and a terminal lid attached to its extremity to keep the biological active composition inside, and the other extremity being the dispenser. The extremity of the dispenser shows an open in order to release the composition in the rumen. This invention deals with a capsule for a slow and gradual release of a biological active composition, not citing any nitrates, thus not colliding with the requirements proposed in the invention presented here.

The patent CA2725380 describes a method which includes a dispenser for ruminant feeding, plus one or more nutritional supplements, in which dispenser is attached a gas analyzer that stays close to the place where the animal introduces its head. The method determines if a specific ruminant accessed the feedbunk (dispenser), by reading the identification of a RFID ear-tag, and also release a nutritional supplement in order to reduce methane. The method includes a gas analyzer to determine the levels of carbon dioxide and methane, also including a data processor that modifies the type and amount of feed offered in the next feeding, in order to control de production of methane and achieve the animal performance desired. This protection is related to a feeding equipment, moving away from the characteristics of the invention proposed here.

The document WO2010071222 reports an inhibitor of ruminal methane emission in ruminants. Precisely, it is an inhibitor of methane emission by ruminant characterized by hydrogen peroxide as the active compound. The innovation is about mitigation of methane production with peroxides, moving away from the characteristics of the invention proposed here.

The patent WO2006040537 is about the inhibition of methane production in ruminants and/or improvement of meat and/or milk production and quality. In particular, this invention makes reference to the use of encapsulated organic acids, especially fumaric acid. It is also contemplated a composition comprehending ruminant feeding, by using encapsulated fatty acids, especially fumaric acid, for utilization in the reduction of methane production by ruminants. Such uses and compositions may also, alternatively, result in a weight gain increase and/or milk production. This protection describes encapsulated organic acids without mention of nitrates, moving away from the characteristics of the invention proposed here.

The patent JP2003088301 demonstrates a composition that inhibits the generation of methane without making the ruminal environment worse, by offering at least one selected strain of Lactobacillus, obtained from sheep milk derived products naturally fermented, yeasts and oligossacharides to a ruminant by oral administration. The inhibitory effect on methane may be improved with nitrate addition, and lactobacillus and yeasts comprises at least one type of microorganism, belonging to Trichosporon, Candida, Leuconostoc, Lactococcus and, in particular, oligossacharides, preferentially, galactoligossacharides. Such invention deals with milk-derived products to inhibit methane production, without mention of encapsulated nitrates, moving away from the characteristics of the invention proposed here.

The patent GB1445560 demonstrates a composed feed, supplemental block, liquid feed supplement, slow-release pellets, ensiled forage, hay or grain containing isobutyraldehyde with a mixture of adipic, glutaric and succinic acid, acetic acid, formol, sulfuric acid or trioxane in order to inhibit the production of methane in the rumen. The use this pelletted diet may contain barley, wheat, peanut, molasse, salt, limestone, bicalcium phosphate. The patent describes only an animal diet, moving away from the characteristics of the innovation proposed here.

### BRIEF DESCRIPTION OF THE DRAWINGS

A more complete understanding of the present disclosure and certain advantages thereof may be acquired by referring to the following detailed description in consideration with the accompanying drawings, in which:

FIG. 1 is a graph displaying nitrate release curves of non-encapsulated and encapsulated calcium nitrate decahydrate.

FIG. 2 is a graph displaying nitrate concentrations of encapsulated nitrate product under solubilization in aqueous medium.

FIG. 3 is a graph displaying nitrate release percentage of encapsulated nitrate product in aqueous medium.

### DETAILED DESCRIPTION OF THE INVENTION

Taking into account the gaps presented in the art, it is proposed, as an innovation, compositions based on nitrates, used as feed additives to reduce the methanogenesis in ruminants combined or not with sulfates, since sulfur-based compounds improve the reduction of nitrate intoxication risks, being this composition coated with vegetable fats that reduce the absorption rate of nitrates by the animal, minimizing the accidental intoxication risks derived from feed management—a problem until this moment without technical solution, demonstrating in such a way its inventive activity. The encapsulation of the aforementioned composition also solves the problems related to nitrate adaptation, higroscopicity, which hampers the transportation and stowage excessively, and related to animal palatability, due to the excessive bitterness of the composition without the mentioned coating.

Such granules, or their variations, are manufactured with nitrates and sulfates, which are responsible by mitigation of methane production, combined with additives or even similar compositions, recovered/encapsulated with hydrogenated vegetable fats, being them responsible by the slow and gradual release/solubilization of nitrates and sulfates in the ruminal environment, with the purpose of avoiding animal intoxication and promoting the complete ruminal metabolism of nitrate and sulfates.

In a similar way, alternatively to coating with vegetable fats, it is possible to use any other material compatible with the animal nutrition that shows equal or similar properties from those presented in fats in terms of resulting in a controlled release of the substance. It is distinguished here natural materials, degradable in the rumen or not, such as cellulose and carboxycellulose-based emulsions (added, as example, calcium carbonate, saccharose, vegetable oils, and xanthan gum), coatings containing starch and other polysaccharides mixed with polyvinyl alcohols, as well as coatings based on lignin/lignosulphonates or chitosan biopolymers.

Alternatively, coating may also be composed of synthetic polymers, degradable in the rumen or not, such as carboxyvinyl; polyacrylic acid (acrylic resins, polyethylenes, etc); alginates; polyhydroxyalkanoates; polyhydroxyoctanoates; polyhydroxybutyrates (Biopols); polycaprolactones; polylactic acids; solutions of biuret with urethane and tungue oil; mixtures of isocyanates with alkydic resins, castor oil and peroxides; mixtures of stearamides with paraffin, magnesium stearate; other resins (polyurethanes, polyolefins, polyesthers, polyepoxides, silicones, polyvinylidene chloride etc, as well as mixtures thereof); alkyl and cycloalkyl amines; paraffins and waxes derived from petroleum. Besides the antimethanogenic property promoted by nitrates and sulfates, the encapsulation drastically reduces the risks of nitrate intoxication, protecting animal welfare and health, thus minimizing risks of loss by intoxication. The scenario of intoxication when using non-encapsulated nitrates is very likely in the practice.

Additionally, it is highlighted that the encapsulation process is able to release the active compounds nitrate and sulfate in a time interval matching the rumen fluid retention time (approximately 6 to 24 h), thus allowing the complete solubilization of these salts in the rumen.

In practice, there are several situations in which encapsulation brings advantages: management errors caused by animal handlers or people involved in animal feeding are very frequent. High amounts of nitrate may be ingested by animals due to lack of attention. The poor preparation of rations, mistakes during ingredient weighting and an inadequate mixture of them are common situations in the field, which may result in high levels of nitrate ingestion by the animals. As a consequence, encapsulation of nitrates and sulfates protects the animals when high amounts of nitrate are ingested by non-adapted animals. In summary, encapsulation ensures animal safety in case of a nitrate overdose.

An additional advantage of coated nitrates and sulfates is the “feed bunk safety” or “feed bunk protection”, an usual term used in the Brazilian livestock sector. If it rains, and offering uncoated nitrate in uncovered feedbunks, there would be a rapid solubilization of nitrate, since this salt is highly soluble in water. This water containing high nitrate concentrations increases the risk of intoxication, because if ingested may result in animal poisoning and death. Therefore, the coating process drastically delays the solubilization of nitrates and sulfates, resulting in animal safety in the situation described above.

The coating process also eliminates the necessity of gradual and progressive adaptation of animals to nitrate, which in practical conditions lasts around four weeks in order to achieve the doses required for adequate methane mitigation. The adaptation phase to nitrate also results in management problems, increasing the time expended during ration preparation and animal feeding, also making the process more complex which, in turn, increases the chance of operational errors. As a consequence, the encapsulation brings a clear advantage, simplifying the animal feeding and allowing the direct offering of nitrates and sulfates in the recommended doses without risks to the animals.

The slow and gradual rumen release of nitrates and sulfates promoted by coating also ensures their complete metabolization in the ruminal environment. This avoids the absorption of nitrate and its intermediate compound—nitrite—by the rumen wall, therefore reducing their concentration in blood circulation.

Consequently, encapsulation allows complete reduction of nitrate to ammonia, which enhances the efficacy of methane mitigation. It is highlighted that nitrate and/or nitrite, if absorbed by rumen wall, will not drain hydrogen, thus reducing the efficiency of methane mitigation.

Moreover, encapsulation reduces or eliminates the circulation of nitrate and/or nitrites in the blood, avoiding their excretion in urine or milk. In high amounts, nitrate is a surface water and groundwater polluter. Although naturally found in milk, high concentrations of nitrate may be potentially dangerous, especially if ingested by neonates and children, also causing the disease called methemoglobinemia.

Another additional advantage promoted by nitrate and sulfate coating is the slow release of NPN in the rumen. The gradual liberation of nitrogen allows the synchronization of carbohydrate degradation and microbial protein synthesis, permitting an adequate and complete amination of NPN. Concomitantly, the use of nitrates as a nitrogen source replacing more traditional sources (e.g. urea) shows as an advantage the maximization of microbial protein synthesis, since energy for microbial growth derived from nitrate reduction is greater than from methanogenesis. The maximization of microbial protein synthesis is crucial for animal performance improvement, because microbial protein is the most important and the best protein source for ruminant nutrition. In addition to nitrogen, the composition containing coated nitrates and sulfates also provides sulfur, calcium, and magnesium to the animal.

The product is composed by nitrates, preferentially between 40% and 97%, more preferentially between 60% and 85%; oils and fats for coating, preferentially between 1% and 40%, more preferentially between 3% and 20%; sulfates, preferentially up to 50%, more preferentially between 0% and 40%; and other additives, preferentially up to 20%, more preferentially between 0 and 10%.

Preferentially, it is used calcium nitrate and magnesium sulfate. Alternatively, it is admitted the replacement of these salts by similar salts or by a combination of different nitrate and sulfate salts.

Nitrates used, as well as sulfates, must be sufficiently soluble in the rumen fluid, being accepted by animals and, consequently, physiologically suitable. Salts cannot carry heavy metals or other minerals in potentially toxic amounts, also attending the requirements of regulatory agencies for products used in animal feeding. Generally speaking, nitrates and sulfates are provided as inorganic salts.

The calcium nitrate is, preferentially, the double salt of calcium ammonium nitrate decahydrate [5Ca(NO₃)₂.NH₄NO₃.10H₂O], however it is not excluded the utilization of other salts, such as calcium nitrate tetrahydrate [Ca(NO₃)₂.4H₂O], calcium nitrate anhydrous [Ca(NO₃)₂], magnesium nitrate [Mg(NO₃)₂.6H₂O], sodium nitrate (NaNO₃), potassium nitraten (KNO₃), ammonium nitrate (NH₄NO₃), cal-urea nitrate [Ca(NO₃)₂.4CO(NH₂)₂], the double salt of ammonium sulfate and nitrate [(NH₄)₂SO₄.3(NH₄NO₃) or (NH₄)₂SO₄.2(NH₄NO₃)], as well as possible variations in the salts cited above due to number or absence of crystallization water. It has already been demonstrated that uncoated/unprotected calcium nitrate, potassium nitrate, sodium nitrate, and ammonium nitrate reduced methane emission in ruminants.

Similarly, it is not excluded here the utilization of mixtures of nitrates, aiming the addition of new properties or even to improve the mitigating effects of final product.

The magnesium sulfate is, preferentially, the monohydrate or anhydrous (MgSO₄.1H₂O ou MgSO₄), however it is not excluded the use of magnesium sulfate heptahydrate [MgSO₄.7H₂O], sodium sulfate [Na₂SO₄ anhydrous, Na₂SO₄.7H₂O and Na₂SO₄.10H₂O), ammonium sulfate [(NH4)₂SO₄], potassium sulfate (K₂SO₄), calcium sulfate (CaSO₄ or 2CaSO₄.1H₂O), zinc sulfate (ZnSO₄ anhydrous or ZnSO₄.7H₂O), ferrous sulfate (FeSO₄.1H₂O, FeSO₄.4H₂O, FeSO₄.5H₂O or FeSO₄.7H₂O), manganese sulfate (MnSO₄ anhydrous or MnSO₄.4H₂O), copper sulfate (CuSO₄ anhydrous or CuSO₄.5H₂O), as well as not mentioned variations in the salts cited above due to number or absence of crystallization water. It has already been demonstrated the effects of sodium sulfate and copper sulfate, as well as magnesium sulfate, in the reduction of ruminal accumulation of nitrite and in the minimization of intoxication risks.

Similarly, it is not excluded here the utilization of mixtures of sulfates or their potential replacers, aiming the inclusion of other properties or even to improve the mitigating effects of final product.

Similarly, in substitution of sulfate it is also not excluded here the use of elemental sulfur, as well as sulfides (as examples Na₂S.9H₂O, CaS, ZnS, K₂S) and sulphites (as examples Na₂SO₃, K₂SO₃, CaSO₃, MgSO₃).

It has already been demonstrated the properties of sulfides and sulphites in the reduction of ruminal accumulation of nitrite and in the minimization of intoxication risks, both in vitro and in vivo. Finally, here it is also considered the use of persulfates (SO₂⁻⁵), thiosulfates (S₂O₂⁻³) e hyposulphites (SO₂⁻²). L-cysteine (anhydrous, monohydrate and chloridrates) can also be included, being one of the sulfur containing aminoacids that has well-known properties in the reduction of ruminal nitrite accumulation and, consequently, in the minimization of nitrate and/or nitrite intoxication in ruminants. Here, it is not excluded the use of metals containing properties that inhibit nitrate reductase, as being demonstrated for sodium tungstate (Na₂WO₄).

In relation to additives that may preferentially be included in the formulation are cited those able to aggregate properties to the final product, such as aromatizers and flavours, natural or synthetics, but not harmful to animals (as examples monosodium glutamate, saccharine, sucrose, dextrose, glucose, guava essences, vanilla etc), antioxidants (such as vitamin C, beta-carotene, BHT—butylated hydroxytoluene, BHA—butylated hydroxyanisole), acidifiers (citric acid, acetic acid, tartaric acid, fumaric acid, malic acid), emulsifiers/stabilizing agents (such as lecithin, xathans, gums, polisorbates, propylene glycol, monostearates etc) and taste enhancers.

It is essentially important to consider the inclusion of anti-wetting and anti-caking agents which, by finality, are able to maintain the fluidity of granules during storage, such as calcium carbonate, starch, microcrystalline cellulose, tricalcium phosphate, silica/silicates, talcum powder, kaolin, calcium stearate etc.

Concurrently, other nutritional additives can also be included aiming at bringing novel properties to the final composition, such as macrominerals, trace minerals, vitamins (for instance A, B₁, B₂, B₃, B₅ B₆, B₇, B₉, B₁₂, C, D, E e K), essential oils (carvacrol, eugenol, thymol, cynamaldehyde, capsaicin, limonene etc), organic acids (lactate, malate, fumarate, aspartate etc), fatty acids (such as CLA—conjugated linoleic acid; myristic acid; anacardic acid; medium-chain fatty acids—capric acid, caprilic acid, caproic acid, lauric acid; as well as omega-6 and omega-3 fatty acids such as alpha-linolenic acid—ALA; eicosapentaenoic acid—EPA; docosahexaenoic acid—DHA; etc), aminoacids (mainly sulfur-containing aminoacids as cysteine and methionine, but also considering histidine, threonine, leucine, isoleucine, tryptophan, phenylalanine, valine, glycine etc), enzymes (cellulases, hemicellulases, amylases, pectinases, xylases, (β-glucanases, phytases, other glucanases etc), buffers and alkalizers (sodium bicarbonate, sodium sesquicarbonate, calcium carbonate, magnesium oxide etc), yeasts (Trichosporon sp., Candida sp., Leuconostoc sp., Lactococcus sp., Candida kefyr, Saccharomyces cerevisiae etc), fungi (such as Aspergillus oryzeae and Aspergillus niger), probiotics and other live microorganisms (Lactobacillus sp. and mainly those that possess nitrate/nitrite reduction activity, such as Selenomonas ruminantium, Veillonella parvula, Wollinela succinogenes, Megasphaera elsdenii, Propionibacterium acidipropionici, Escherichia coli W3110; and intestinal bacteria, coryneform bacteria, Bacillus subtilis, Methylophilus sp., and Actinomyces sp).

It can also be included galactoligosaccharides and/or nisin, substances known by their properties in the reduction of nitrite accumulation and risks of nitrate poisoning. Finally, other additives potentially usable are antibiotics normally utilized in ruminant nutrition (ionophores—sodium monensin, salinomycin, lasalocid, narasin—other antibiotics such as virginiamycin, avilamycin, bacitracin, flavomycin, tylosin), natural substances with antimicrobial properties (propolis, beta-acids, alfa-acids, other hop-derived acids, cardanol, cardol, tannins, saponins), anthelmintic, and anticcocidials/coccidiostats.

The granules are coated preferentially with vegetable fats, which are responsible for the slow and gradual release/solubilization of nitrates and sulfates in the ruminal environment, in the sense of avoiding animal intoxication and maximizing their complete metabolism in the rumen.

The coating is, by itself, hydrophobic and allows the slow and gradual solubilization of nitrates/sulfates salts. The coating of granules permits the synchronization of nitrates/sulfates release and reduction reactions, in the way of avoiding rumen accumulation of nitrate/nitrite, thus reducing the risks of animal poisoning. The gradual nitrate release permits the reduction of nitrite to ammonium occurring in a similar rate of reduction of nitrate to nitrite, thus avoiding the ruminal accumulation of nitrite. As an additional advantage, encapsulation with fats is biodegradable. Lipids are digested in the small intestine, also serving as a supplemental fat, therefore, providing additional energy.

When coated, granules of final product have 1.5 mm to 12 mm of diameter. The liberation rate of nitrates/sulfates varies between 1% to 30% per hour, more preferentially between 5% to 25% per hour. Considering the density of the final product, it varies between 0.85 g/cm³ to 1.15 g/cm³, more preferentially between 0.90 g/cm³ to 1.10 g/cm³.

The product is destined to all ruminant animals, either domestic or wild species. For instance, here are included cattle, sheep, goat, buffalos, cervids, camelids, giraffids, antelopes, bisons, and yaks. However, by convenience and importance, the technology here described is destined mainly to domestic species such as cattle, sheep, goat, and bubalines.

It is necessary a functional rumen in these animals, being excluded the utilization in pre-ruminant animals, being examples new-born calves and lambs. Additionally, the product is destined to feedlot animals as well as animals on pasture receiving supplementation.

The period of feeding is undetermined, being offered continuously since the moment that the animal possess a functional rumen until the moment of slaughtering. The product has a long-term effect on methane mitigation, without loss of efficiency due to prolonged utilization.

The product is offered in feed (by spontaneous animal ingestion), being a total mixed ration (TMR; mixture of all ingredients required by the animal, such as roughages/forages, concentrates/cereal grains, mineral supplements, vitamin supplements, and additives), protein supplement, energy supplement, protein/energy supplement, or mineral supplement. Such supplements are generally fed to ruminants kept on pasture, being either a high-intake or low-intake supplement, preferentially a high-intake supplement. High and low intake supplements are terms generally used by professionals to designate mixtures of feeds ingested in high (2 g to 4 g per kg of body weight) and low (up to 1 g per kg of body weight) amounts, respectively.

Mixed in ration or supplement, granules of nitrates and sulfates composition can also be fed on top, which means that granules can be dispersed on the top of ration placed in feed bunk. It is also considered the isolated offering of the product, as long as the animal shows spontaneous preference.

The product can be mixed in the ration or supplement at the moment of animal feeding.

Similarly, the product can be mixed in rations and supplements produced by feed companies and feed mills, being in that way stored for long periods of time. Due to its good abrasion resistance, in the moment of mixing, such process can be performed both manually and using mixing wagons.

The coating promotes protection against the high hygroscopicity naturally showed by nitrate salts. Exposed to air and heat, non-encapsulated nitrate absorbs air humidity and liquefies rapidly. Consequently, the encapsulation allows the premixture of the product with rations or supplements, allowing a prolonged storage without quality loss of the final product.

In addition, the encapsulated product containing nitrates and sulfates permits a more homogenous mixing. Nitrate is generally found in a granular form, while sulfate is a powder salt. This granulometric and density variation results in problems related to the adequate homogenization and particle segregation during transport and storage. The encapsulated product containing nitrates and sulfates presented as a single granule has the advantage of minimizing these problems.

In order to prove the effects of this innovation, it was conducted an in vitro trial to measure the release of encapsulated and non-encapsulated nitrate forms, aiming at demonstrating the efficacy of two encapsulation methods with fats when comparing with non-encapsulated nitrate. The material used was calcium ammonium nitrate decahydrate.

In this trial, three treatments were used as follow:

i. Control: non-encapsulated calcium nitrate;

ii. Prototype 1: encapsulated calcium nitrate;

iii. Prototype 2: encapsulated calcium nitrate.

Three replicates were used per treatment. In each 1-L flask, 500 mL of distilled water were added with 2.482 g of calcium ammonium nitrate decahydrate. Prototypes were included in an amount corresponding to 2.482 g of pure calcium ammonium nitrate.

The incubation was performed in a circulation-forced incubator at 39 degree C. and 100 rpm. Samples were collected following treatment additions at 0 min, 5 min, 10 min, 15 min and 30 min; 1 h, 2 h, 4 h, 8 h, 16 h, 24 h, and 48 h. In each sampling time, 5 mL were collected.

The water-solubilized nitrate was analyzed according to the colorimetric method with phenol disulphonic acid following by alcalinization with sodium hydroxide.

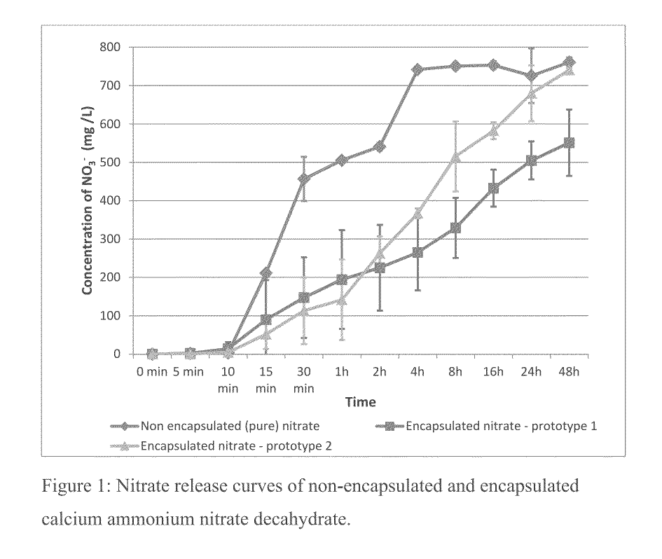

The trial results are showed above in FIG. 1, where is presented the nitrate release curves of non-encapsulated and encapsulated forms of calcium nitrate decahydrate, demonstrating that encapsulated nitrate sources presented a slower solubilization when compared with the non-encapsulated source. This supports that encapsulation with fats is effective and provides a slow and gradual nitrate release in aqueous medium. Therefore, coating of nitrate granules brings as an advantage the reduction of animal intoxication risks.

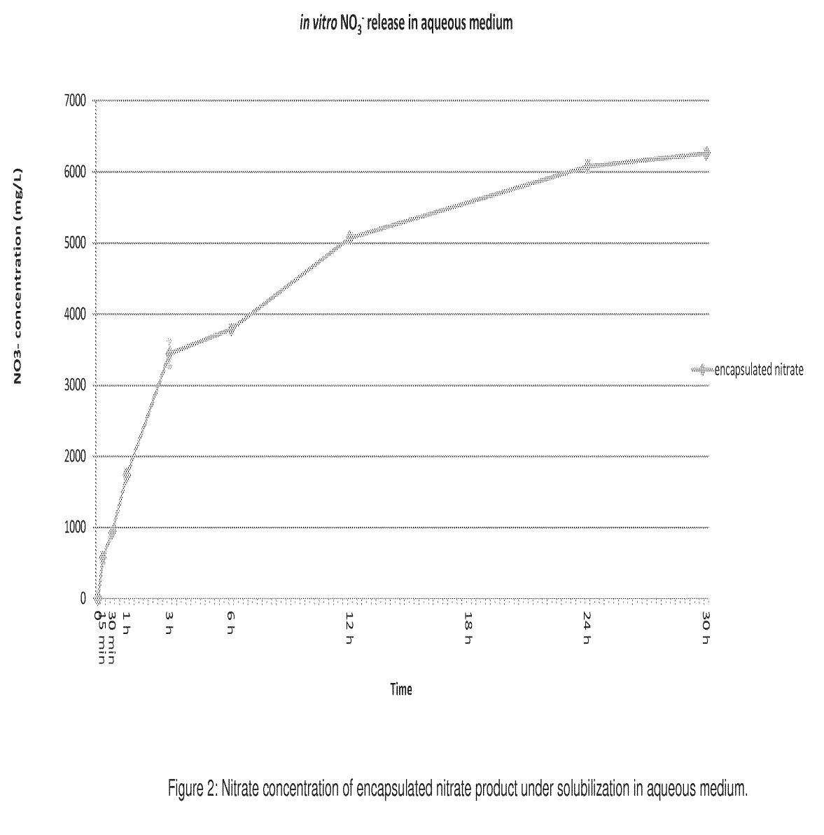

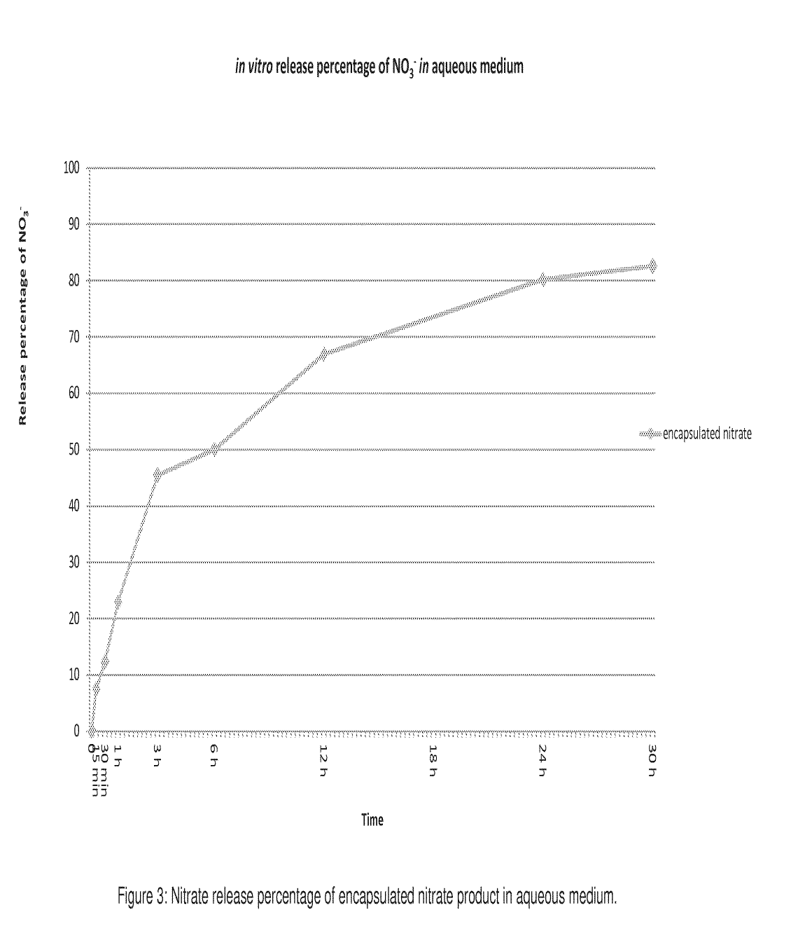

Another trial was performed with the objective to evaluate the in vitro release curve of nitrate in a nitrate-encapsulated product. Samples were obtained over time from an aqueous solution containing the encapsulated salt. Nitrate concentration was analyzed in each sample, that way obtaining the profile of the nitrate release curve, thus illustrating the properties of the encapsulation proposed here. With the results expressed in FIG. 2, it is noticed the presence of nitrate release in aqueous medium over time, whilst FIG. 3 shows the percentage of salt released over time. Such results demonstrate that the purpose of the innovation here described fulfill its role.

This innovation is not limited to the representations here mentioned or illustrated, must being comprehended in its broad scope. Many modifications and other representations of this innovation will come up in the mind of those skilled in the technique in which this innovation belongs, having the benefit of teaching presented in the previous descriptions and sketches attached. Besides that, it must be understood that this innovation is not limited to the specific form revealed, and modifications and other forms are comprehended as included inside the scope of the attached claims. Although specific terms were used here, they are employed only as a generic and descriptive form and not with a purpose of limitation.

1. A method comprising:
reducing methane emission in one or more ruminant animals and simultaneously avoiding nitrate intoxication in the one or more ruminant animals, by feeding the one or more ruminant animals a feed comprising a slow-release ruminant feed additive;
wherein the slow release of nitrate ion from the feed additive keeps the overall levels of released nitrate in the rumen below a level that would result in nitrate intoxication of the one or more ruminant animals, yet provides a level of non-protein nitrate ion to ruminant bacteria high enough to act as an antimethanogenic agent, resulting in a reduced production of methane; and
wherein the slow-release ruminant feed additive is in a granular or round form encapsulated by a coating, and comprises:
i. 40% to 85% in weight of a source of nitrate ion, wherein the source of nitrate ion is physiologically suitable for ruminant animals;
ii. 0% to 50% in weight of a source of sulfur, wherein the source of sulfur is physiologically suitable for ruminant animals;
iii. 1% to 40% in weight of the coating, the coating comprising one or more hydrogenated vegetable fats; and
iv. 0% to 20% of additives in weight;
wherein the nitrate ion source, sulfur source and any additives are combined and presented in granular or round form, and are encapsulated by the coating; and
wherein, once the feed additive is in a rumen of the one or more ruminant animals, the nitrate ion once consumed is slowly released from the feed additive such that the overall levels of released nitrate in the rumen are maintained below a level that would result in nitrate intoxication of the one or more ruminant animals, yet provide a level of non-protein nitrate ion high enough to act as an antimethanogenic agent, resulting in a reduced production of methane.

2. The method of claim 1, wherein the source of nitrate ion comprises calcium nitrate tetrahydrate [Ca(NO₃)₂.4H₂O], calcium nitrate anhydrous [Ca(NO₃)₂], magnesium nitrate [Mg(NO₃)₂.6H₂O], sodium nitrate (NaNO₃), potassium nitrate (KNO₃) and ammonium nitrate (NH₄NO₃), cal-urea nitrate [Ca(NO₃)₂.4CO(NH₂)₂], the double salt of ammonium sulfate and nitrate, or a combination thereof.

3. The method of claim 1, wherein the source of sulfur comprises magnesium sulfate heptahydrate [MgSO₄.7H₂O], sodium sulfate, ammonium sulfate [(NH₄)₂SO₄], potassium sulfate (K₂SO₄), calcium sulfate, zinc sulfate, ferrous sulfate, manganese sulfate, copper sulfate, cysteine, one or more sulfides, one or more sulphites, elemental sulfur, or a combination thereof.

4. The method of claim 1, wherein the additive comprises one or more natural or synthetic aromatizers, flavors, taste enhancers, antioxidants, acidifiers, or a combination thereof.

5. The method of claim 1, wherein the additive comprises one or more natural or synthetic emulsifiers, stabilizing agents, anti-wetting agents, anti-caking agents, or a combination

6. The method of claim 1, wherein the additive comprises one or more natural or synthetic vitamins, macrominerals, trace minerals, or a combination thereof.

7. The method of claim 1, wherein the additive comprises one or more natural or synthetic organic acids, fatty acids, amino acids, or a combination thereof.

8. The method of claim 1, wherein the granular or round form of the combined nitrate ion source, sulfur source and any additives, has a shape that is approximately spherical with 1.5 mm to 12 mm of diameter, with a density varying from 0.85 g/cm³ to 1.15 g/cm³.

9. The method of claim 1, wherein, when the additive is in a rumen of ruminant animal, the additive has a liberation rate of any nitrate ions and sulfur in the additive into the rumen varying from 1% to 30% per hour, with the liberation rate determined by an in vitro technique with distilled water, with incubation time varying from 0 to 48 hours at 39° C. under circulation.

10. The method of claim 1, wherein the source of nitrate ion comprises calcium nitrate.

11. The method of claim 10, wherein the source of nitrate ion comprises the double salt of calcium ammonium nitrate decahydrate [5Ca(NO₃)₂.NH₄NO₃.10H₂O].

12. The method of claim 1, wherein the source of sulfur comprises magnesium sulfate.

13. The method of claim 12, wherein the source of sulfur comprises monohydrate or anhydrous magnesium sulfate (MgSO₄.1H₂O or MgSO₄).

14. The method of claim 1, wherein the additive comprises one or more yeasts, fungi, probiotics, antibiotics, natural antimicrobials, anthelmintic agents, anticcocidials, coccidiostats, or a combination thereof.

15. The method of claim 1, wherein the additive comprises one or more enzymes, buffers, alkalizers, or a combination thereof.

16. The method of claim 1, wherein the feed additive comprises 3% to 20% in weight the source of sulfur.

17. The method of claim 1, wherein the feeding the one or more ruminant animals the feed comprising the slow-release ruminant feed additive is performed without any prior adaptation to nitrate by the one or more ruminant animals.

18. The method of claim 1, wherein, when the additive is in a rumen of ruminant animal, the additive has a liberation rate of any nitrate ion and sulfur in the additive into the rumen varying from 5% to 25% per hour determined by an in vitro technique with distilled water, with incubation time varying from 0 to 48 hours at 39° C. under circulation.
